(19) United States
(12) Patent Application Publication
(10) Pub. No.: US 2026/0229332 A1
(43) Pub. Date: Aug. 6, 2026

(54) THREE MODULE ELECTRONIC MEDICATION ADMINISTRATION RECORD VITAL DETECTION AND ALERT SYSTEM

(71) Applicants: Fabian Franco, Kerrville, TX (US); Tracy Allen Byerly, II, Kerrville, TX (US); Joel Thomas Bennett, Kerrville, TX (US)

(72) Inventors: Fabian Franco, Kerrville, TX (US); Tracy Allen Byerly, II, Kerrville, TX (US); Joel Thomas Bennett, Kerrville, TX (US)

(73) Assignee: Tres & Associates, LLC, Kerrville, TX (US)

(21) Appl. No.: 19/042,187

(22) Filed: Jan. 31, 2025

(51) Int. Cl. G16H 20/10; A61B 5/00; G16H 40/40

(52) U.S. Cl. CPC G16H 20/10; A61B 5/681; A61B 5/746; G16H 40/40

(57) ABSTRACT

A three module Electronic Medication Administration record (eMAR) vital detection and alert system with associated hardware is disclosed. The eMAR system comprises an eMAR module in communication with and Activities for Daily Living module and a Vitals Module to track and administer medications timely, record daily activities residents perform, and monitor deviations of vital signs of a resident constantly and in real time. A wearable vitals detection device is worn by or affixed to a resident and communicates vitals data twice a day to the Vitals Module to establish rolling seven day baseline vital signs for the resident, and continuously records vital signs for each resident. The Vitals Module executes visual cue, alert notifications and text messages to the medical care individuals when it receives vitals data from the wearable vitals detection device that deviates from the baseline within predefined parameters.

### BACKGROUND

### 1. Field of the Invention

The present invention relates to an electronic medical administration record (eMAR) system, and more particularly to an eMAR system comprising three modules. The eMAR system of the present invention is in communication with an Activities for Daily Living (ADL) module and a vitals module, which calculates a seven day rolling average of vital signs of a resident to establish a baseline and automatic alert system when vital signs vary within predefined parameters from the baseline.

### 2. Description of the Related Art

eMAR systems are broadly designed to be mobile medication administration systems which enabled the administration of medication to residents of a facility, while tracking medications, including the ordering and distribution of medications within a long-term-care facility, nursing home, or other medical setting. Many of the prior art eMAR systems claim to streamline the medication administration process, reduce medication errors and user error by a MED Tech while making the administration of medicine to a resident or patient efficient.

eMAR systems of the prior art claim to send customized notifications to necessary personnel when medications are either missed or administered. Some eMAR systems in the prior art streamline documentation, and include integration of pharmacies for messaging between the assistant living facility and the pharmacy for efficiency in filling medications. These eMAR systems provide improved resident record management, oversight, and medication administration. However, most of the eMAR systems of the prior art focus, in large part on integrating patient medical records in medicine administration with interconnectivity to associated pharmacies, but do not provide further patient care, such as monitoring the baseline vital statistics of a resident to determine health parameters indicative of medical danger to a resident, such as parameters indicative of cardiovascular, stroke, heart attack danger, or risk of falling of a resident.

Other systems in the prior art attempt to track vitals of residents within the eMAR system to monitor various parameters of the resident's health. However, these modules have very little integration within the overall eMAR structure and do not have the ability to perform real-time alert and tracking of vital signs of the residents. Moreover, the limited vitals recording modules and function in the prior art require manual graphic user interface by a MED Tech to input the vitals data into the eMAR system, without the possibility of automation. Thus, every vital sign recorded by systems of the prior art require several steps to be executed by a MED Tech, such as recording times to take the vital signs, physically and accurately taking the vital signs, and manually imputing vital signs into the eMAR systems of the prior art. Furthermore, no eMAR systems of the prior art calculate rolling seven day baseline computations of vital signs to establish baseline vitals for residents. Prior art eMAR systems also do not establish variable Ranges to track significant deviations in any given vital sign for a resident and automatically alert the medical staff, through the system when significant deviation is observed in the system. Prior art eMAR systems also do not have the ability to continuously monitor vital signs through hardware devices work on attached to the resident and in communication with the system.

It is desirable to create an eMAR system that integrates other modules to form a seamless long-term facility care system that allows a Director of Nursing (DON), other administrators or users, and/or PCA's to seamlessly streamline care for the resident by integrating an eMAR module with an ADL module to allow the DON and the PCA's to work seamlessly together to monitor the daily activities the resident can, has, or cannot and has not performed during daily rounds at the facility.

It is further desirable to develop an eMAR system that integrates and communicates with a vitals module, wherein the vitals module monitors various parameters of the resident's health to determine when a patient is at a health risk, or a risk of falling. It is further desirable to develop an eMAR system that is integrated with and in communication with both an ADL module and a vitals module, which are integrated and in communication with each other to provide an integrated system for administration of medicines, monitoring of activities for daily living and monitoring the vital statistics of the resident. It is further desirable for the system to run dependencies between the eMAR module, ADL module and vitals module to improve communication between the three modules within the system. It is further desirable to integrate a communicative hardware wearable device for placement on the resident to communicate with the vitals module and send vital sign information from the resident to the vitals module at predetermined intervals.

### SUMMARY OF THE INVENTION

As used herein, the term “facility” is desired to mean a long care term facility or facilities, nursing home, assisted living facility or facilities, or any other care facility whose purpose is the long-term care of a resident, including the administration of medication and the monitoring of the resident through the collection of medical data at predetermined intervals. In such facilities, the biggest risk to a resident is often the risk of falling, which can lead to injury, hospitalization, and even death. For any given resident, there are as many as seven different vital signs that, when systematically monitored alone or in combination may be indicative of increased risk of fall, or risk of other medical complications such as infections or injury to the skin and soft tissue system, infections of the urinary system/tract, infections of the respiratory system/tract, as well as other infections. Yet, facilities mostly do not systematically record data on the vital signs of a resident to analyze and hopefully prevent such accidents or medical complications before they occur. Facilities further do not systematically record vital signs from a resident to establish a baseline for each vital of each resident, thereby establishing a constant to measure any variability against.

The present invention is different than the prior art. The present invention, in its most comprehensive form comprises an eMAR module in communication with ADL module and a vitals module, which are in communication with each other, along with a hardware device attachable to a resident, which is in remote communication with the vitals module. However, the present invention is also designed to be modular, allowing for any combination of modules to be arranged within a facility to meet the needs of that facility. For instance, if the facility has a pre-existing eMAR system, a vitals module may be integrated therewith, along with a wearable hardware device if desired. If a facility does not desire an ADL module or a vitals module, the eMAR module may be installed within the facility and operate independently.

The system of the present invention may be modulated to meet the needs of any given facility. Each module of the present invention may be installed at any facility as a stand-alone product, or in any combination with another module. In this regard, each module is fully functional on its own, or may be integrated with other modules.

Regardless of the configuration within a facility of the system of the present invention, the system of the present invention will have a facility core level within the system. The facility core level is the base of a layered architecture, which provides the architecture for the modules to integrate within the facility core level and share access to facility information, resident information and user information or staff information. The facility core enables communication between the modules, allowing information stored in each module to be communicated intermodularly. Dependencies are run in between the various modules of the system to allow communication and determine variants of information, if any, providing access to information stored in each module by other modules.

Each module of the system of the present invention comprises its own feature sets and operability within each individual configuration. Integration of multiple modules activates the dependencies between them and will allow the sharing of information at the facility level. For instance, the eMAR module stores the medication information for each specified resident, along with the resident information. eMAR module stores medication schedules, medication alert commands and notifications, and memory to record data entered by the MED Tech regarding the administration of medication, as well as alert commands when scheduled medications haven't been administered, or have not been administered timely.

In this regard, the eMAR module of the present invention can schedule medications as well as allow for the change medication doses, addition, editing or deletion of medications for any specified resident within the eMAR module. The eMAR module also has stored within it a data set for each resident for PRN medications. Medication administration alert and notification commands within the eMAR module are associated with a notification command to notify a DON of the facility when the scheduled medications haven't been administered or were administered late.

In embodiments where the eMAR module and the vitals module are installed in a facility and are in communication with one another, in addition to the functions of the eMAR module, the vitals module captures and records the vitals of each resident, and associates vital dependent medications with each resident. The vitals module calculates rolling averages of the vitals of each resident to determine a baseline for each vital for each resident. The collection of the vital data is set up for each resident on a predetermined time parameter, most typically every day. However, the collection of data can be varied within the vitals module as needed for each resident. Within each vital collection command, date and time commands within the vitals module will allow the vitals data to be date and time stamped within the system. As will be explained in more detail hereinbelow, alert commands are created to alert DON's or PCA's, or other users of the system when any particular vital sign of any particular resident reaches beyond their control limits.

In the preferred embodiment, a resident is equipped with a hardware wearable device which records all vital signs of a resident on the predetermined time basis, and through the hardware on the wearable, communicates the data recorded to the vitals module, which communicates the same data to the resident page within the eMAR module for recordation and calculation.

In embodiments where the eMAR module is in communication with the vitals module as well as the ADL module, the ADL module will communicate with the eMAR module to record ADLs for each resident, and send notifications to the DON if ADLs haven't been recorded. The ADL module also can record if a resident is out of the facility and the reason for the absence, such as situations where the resident is in the hospital, or needs to attend a doctor's appointment.

The ADL module of the present invention is programed to collect ADL records, generate ADL reports and issue notifications to the DON for any missing ADL records. In order to input into the ADL module, a PCA, through a graphic user interface on a device such as an iPad, tablet or other mobile device, will input the ADL data into the ADL module. The ADL module, through the login information will capture which PCA recorded the ADL, timestamp and date are recorded upon input by the PCA. The ADL will capture the PCA's daily notes, and will issue reports upon demand by the DON.

### BRIEF DESCRIPTION OF THE DRAWINGS

FIG. 1 is a flowchart overview of the system of the present invention.

FIG. 2 is a flowchart of the levels of the present invention from the administration level to the corporate level and facility level, and the communication between the modules of the present invention in the dependencies between the modules.

FIG. 3 is a flowchart of the programing and integration of the system of the present invention within a facility or group of facilities

FIG. 4 is a flowchart of the eMAR module and the steps for inputting data in the eMAR module as well as the steps performed by the eMAR module of the present invention.

FIG. 5 is a flowchart of the vitals module including the steps for inputting data in the eMAR module as well as the steps performed by the eMAR module of the present invention.

FIG. 6 is a flowchart of the ADL module including the steps for inputting data in the eMAR module as well as the steps performed by the eMAR module of the present invention.

FIG. 7 is a schematic of a resident's screen as encountered through a graphic user interface by the PCA.

FIG. 8 is a schematic of a resident's screen as encountered through a graphic user interface by the PCA.

FIG. 9 is a schematic of a resident's screen as encountered through a graphic user interface by the PCA.

FIG. 10 is a schematic of a resident's screen as encountered through a graphic user interface by the PCA.

FIG. 11 is a schematic of a resident's screen as encountered through a graphic user interface by the PCA.

FIG. 12 is a schematic of a resident's screen as encountered through a graphic user interface by the PCA.

FIG. 13 is a schematic of a resident's screen as encountered through a graphic user interface by the PCA.

FIG. 14 is a graph showing an example of a resident baseline for body temperature, showing Range 1 alerts, Range 2 alerts and Range 3 alerts as contemplated by the interaction between the wearable and the vitals module. The graph indicates by color when a Range 2 alert is triggered, which will send a visual cue to the Residents page 100 and notifications to the MED Tech and DON, and when Range 3 alerts have been triggered, which thereby triggers the vitals module to send an alert command to the eMAR module and alert the DON via alert command and text message.

FIG. 15 is a graph showing an example of a resident baseline for respiratory rates, showing Range 1 alerts, Range 2 alerts and Range 3 alerts as contemplated by the interaction between the wearable and the vitals module. The graph indicates by color when a Range 2 alert is triggered, which will send a visual cue to the Residents page 100 and notifications to the MED Tech and DON, and when Range 3 alerts have been triggered, which thereby triggers the vitals module to send an alert command to the eMAR module and alert the DON via alert command and text message.

FIG. 16 is a graph showing an example of a resident baseline for heart rate showing Range 1 alerts, Range 2 alerts and Range 3 alerts as contemplated by the interaction between the wearable and the vitals module. The graph indicates by color when a Range 2 alert is triggered, which will send a visual cue to the Residents page 100 and notifications to the MED Tech and DON, and when Range 3 alerts have been triggered, which thereby triggers the vitals module to send an alert command to the eMAR module and alert the DON via alert command and text message.

FIG. 17 is a graph showing an example of a resident baseline for blood pressure, showing Range 1 alerts, Range 2 alerts and Range 3 alerts as contemplated by the interaction between the wearable and the vitals module. The graph indicates by color when a Range 2 alert is triggered, which will send a visual cue to the Residents page 100 and notifications to the MED Tech and DON, and when Range 3 alerts have been triggered, which thereby triggers the vitals module to send an alert command to the eMAR module and alert the DON via alert command and text message.

FIG. 18 is a linear graph of oxygen saturation of a resident, showing Range 1 alerts, Range 2 alerts and Range 3 alerts as contemplated by the interaction between the wearable and the vitals module. The graph indicates by color when a Range 2 alert is triggered, which will send a visual cue to the Residents page 100 and notifications to the MED Tech and DON, and when Range 3 alerts have been triggered, which thereby triggers the vitals module to send an alert command to the eMAR module and alert the DON via alert command and text message.

### DETAILED DESCRIPTION

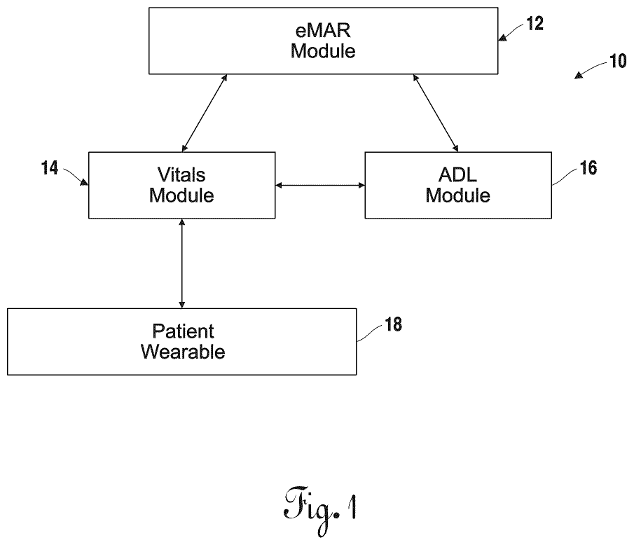

Referring to FIG. 1, a complete module interaction of the present invention 10 is disclosed. It should be understood that in any given facility each module could operate as a stand-alone module, two modules could work in conjunction with one another without a third, or all three modules could be installed and in communication with one another. If a facility has a pre-existing eMAR module, and only desired a vitals module, vitals module 14 could be installed and could operate on its own to alert the DON during the vitals operating procedure. Likewise, if an ADL module is all that is desired by any given facility or group of facilities, ADL module 16 can work independently of any other module to record daily living activities of a resident.

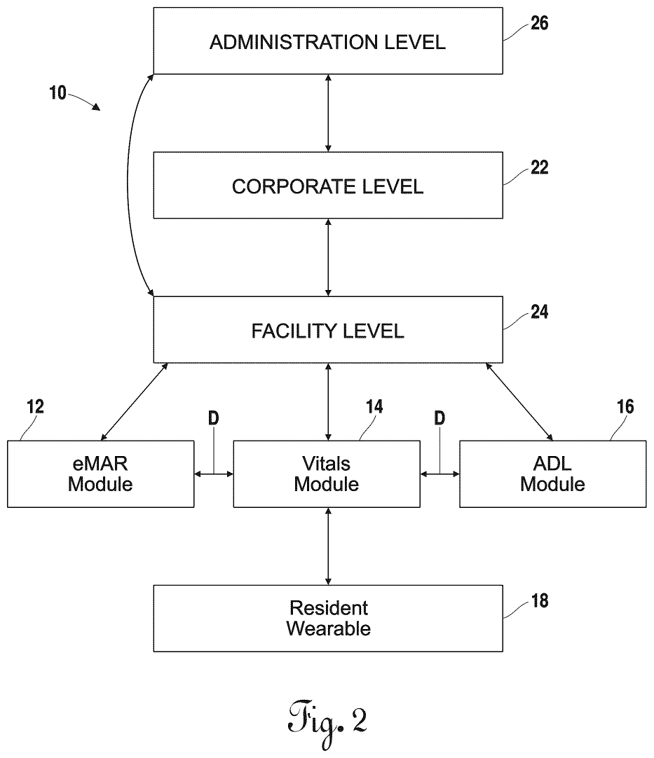

Referring to FIGS. 1-3, as a full installation of the present invention 10, eMAR module 12 is installed at a given facility at the facility level 24 by an installation on the administration level 26. Optionally, a corporate level 22 may exist in a scenario when a corporate entity owns several facilities, and desires to install the present invention 10 across several facilities. See FIG. 2. In such a situation, the administrator (not shown) installs the system of the present invention 10 at the corporate level 22, which customizes the installation throughout the facilities owned and/or operated by the corporate entity.

Returning to FIG. 1, in an installation where all modules and hardware of the present invention 10 are desired, eMAR module 12 is installed at the facility, and vitals module 14 is installed to be in communication with eMAR module 12. The communication of the various modules is established through dependencies within the system, allowing the sharing of information between each module. ADR module 16 is installed to be in communication with vitals module 14 and eMAR module 12, again establishing a dependencies between vitals module 14 and ADL module 16 and eMAR module 12.

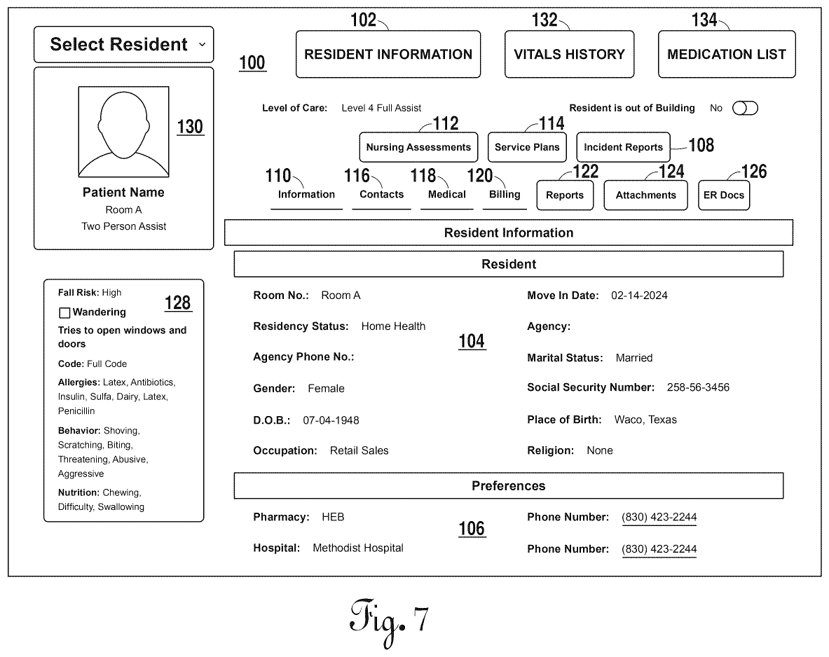

Patient wearable 18 is a hardware device which is designed to be worn by a resident to automatically detect the vital signs of any given resident on a constant basis. Furthermore, the wearable 18 sends vital sign information of the resident to the vitals module 14 on a predetermined time basis to establish rolling seven day baselines for each vital sign for each resident. For instance, in the present system 10, resident vitals are taken via patient wearable 18 and communicated to vitals module 14 every twelve hours. Vitals module 14 sends the vitals for each resident to the eMAR module for recording on the resident's page 100 (see generally FIGS. 7-13), as explained in more detail hereinbelow. Wearable 18 continuously detects the vital signs of the resident, and sends vital sign information to vitals module 14 when a deviation with in Range 2 for any given vital is detected. Vitals module communicates this deviation to the resident's page 100 and displays a visual cue and alert to alert the MED Tech of the deviation. Moreover, when a Range 3 deviation is detected, the wearable 18 sends the vital information to vitals module 14, which sends alerts within the system 10 to the DON and the MED Tech, as well as text alerts to the DON and Med Tech. Although described as a wearable that is attached to a resident similar to a wrist watch, it should be noted that wearable 18 can also be integrated with the resident in any form such as adhesive, cutaneous insertion, or any other available method so long as wearable 18 is able to continuously record the vital signs of the resident.

In this manner, wearable 18 (a) records the vitals twice a day (for instance 8 a.m. and 8 p.m.) to create the seven day rolling baseline for the resident, while (b) continuously detecting the vital signs of the resident, while (c) sending vital information for Range 2 deviations to allow vitals module 14 to alert the MED Tech via visual cue of such deviation and (d) sending vital information for Range 3 deviations to allow vitals module 14 to alert the MED Tech and DON via visual cue, alert within system 10, and via text message.

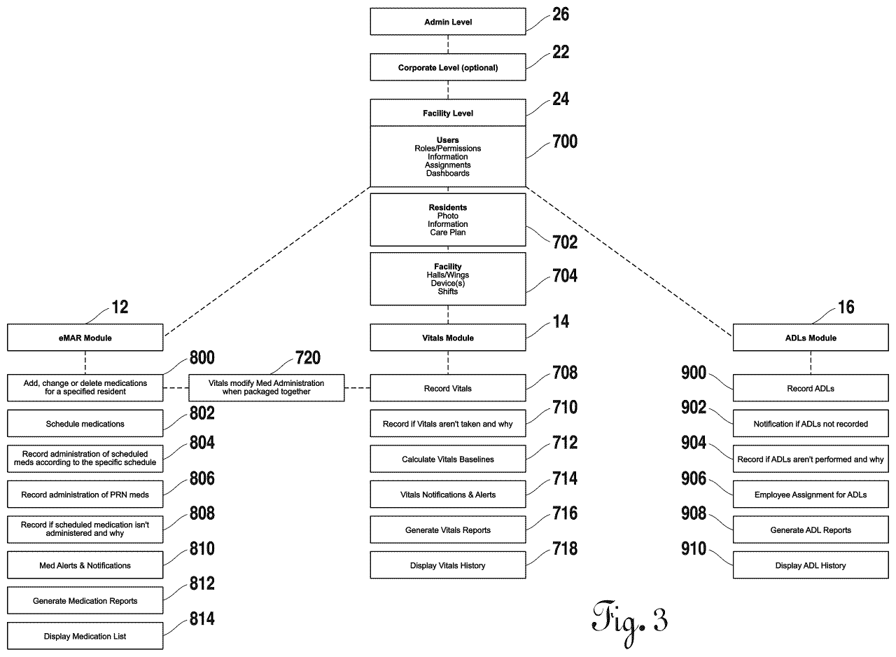

Referring to FIG. 3, the overall data input, programming and implementation of the system of the present invention 10 is disclosed. The admin level 26 is, in the preferred embodiment the level of the present invention 10 where the administrator, or product provider accesses the system to implement the system within a facility. In a setting where a corporate entity owns several facilities, and desires implementation across several facilities, corporate level 22 implementation may be utilized. Admin level 26 is in communication with facility level 24, and the administrator, through admin level 26 will enter the users desired at any given facility. The users will generally be given designations such as the DON, PCA's, or other users. User programming 700 will include assigning roles and permissions to information, information about the user, as well as task assignments and dashboards. Implementation at the admin level 26 further includes input 702, for inputting residential information.

The residential pages 100 of each resident are input during the input step 702, to include a photograph of the resident, pertinent medical information, and care plans. The eMAR module 12 of the present invention is further configured through admin level 26 to designate facility halls, wings and rooms. This is done during facility programming step 704. Within each hall, wing, or residence, devices will be present at the resident's room will be listed, and shifts will be assigned for PCA's and/or other users.

Programming and data upload of eMAR module 12 includes programming step 800 which allows the administrator or designated administrator at the facility level 24 to add, change or delete medications for a specified resident 800. Any modification of medications for residents may automatically modify the medicine administration that is packaged with the recorded vitals 708 through automated command 720. The administrator performs programming step 802 to schedule the medications, and the eMAR module 12 will record the administration of scheduled medicines according to the specific schedule, as input by the PCA through graphic user interface. The recording of the administration of the scheduled medications is performed in step 804.

Step 806 further allows a PCA to administer PRN medications and record the same through graphic user interface into the eMAR module 12. eMAR module 12 will execute an alert command and notification 810 if medicines are not timely administered to resident, or are not administered at all. The administrator may execute report commands 812 to generate reports for any given resident. Medication lists input by the administrator into the eMAR system are displayed on the display medication lists 814 of a resident's page 100 so that the PCA can see the list of medications to be administered, and the times therefore.

Vitals module 14 is designed to record the seven-day rolling average of vital statistics 708. If the vital statistics are not automatically recorded and transmitted via the wearable 18 to vitals module 14, an alert command is executed within vitals module 14, and the vitals module 14 records that the vitals have not been taken during recording step 710.

Calculation step 712 allows the vitals module 14 to calculate a seven-day rolling average or baseline of the vital signs of any particular resident. Baseline provides a constant against which variables are measured to determine whether vitals module executes a range two or range three alert when deviation from the baseline is detected within a resident. A DON or administrator may generate vitals reports by executing an unassociated command 716. The vitals history of a resident is displayed 718 on resident's page 100.

ADL module 16 provides for graphic user interface for a PCA to input within ADL module 16 the ADLs performed or not performed by resident. The PCA records ADL 900. A notification command 902 is executed if any ADL is not recorded. The PCA records any ADLs that are not performed and states the reason why step 904. An administrator DON can execute or generate ADL report 908 to generate ADL reports for any given resident. The history of the ADL of a resident 910 is displayed on resident page 100.

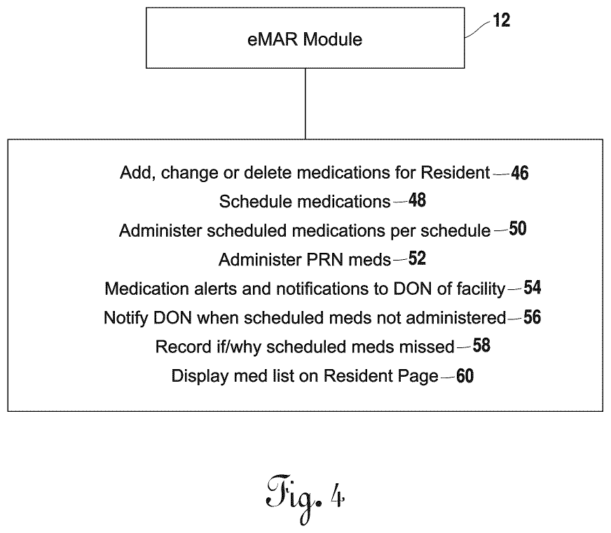

Referring to FIG. 4, eMAR module 12 is installed, and comprises the medication information for each resident of a facility. The eMAR module 12 stores the information on each resident's individual resident page 100 within the system 10. For each resident, eMAR module 12 provides command steps to add, change or delete medications for residents 46. A separate step within the system 10 is a schedule medications command 48. Once medications of each resident are scheduled, a command is executed to administer scheduled medications for schedule 50. There is a separate command within the eMAR module 12 to administer PRN meds 52. Another step or command within the eMAR module 12 is to issue medication alerts to the PCA whenever a medication is due, and a separate notification command to notify the DON of a facility 54, when the medication is not administered, or not administered timely.

When a medication is not administered or is late to be administered, eMAR module 12 issues a notification command 56 to notify the DON that the medication was not administered, or was late to be administered. The PCA, through graphic user interface of a tablet, or other smart device in communication with the system 10 inputs if the medication was missed, and why, which is recorded within the eMAR module 12 on that particular resident's resident page 58. Finally, the eMAR module 12 records the medicines for each resident on the resident's page 100, where it is displayed through graphic user interface to the PCA, DON or other user 60.

As such, the eMAR module 12 is designed to give the user, whether PCA, DON, or other user the ability to view medications, whether scheduled or PRN, within their scheduled time window, to administer the medications as scheduled. If the medication is not administered, or is late, the eMAR module 12, through the command steps, issues an alert to the DON. These alerts reduce the risk of medications being missed via human error, thus reducing an added risk injury to residents.

The eMAR module 12 of the present invention 10 is designed to store the geographic layout of each facility. The eMAR module 12 of the present invention 10 stores a geographic layout of each facility. Typically, a hall is identified by name or alphabetical letter and/or number designation, or any other identification moniker desired. Each room within each hall is identified to correspond to the room identification designation of the facility, whether numeric, alphanumeric, or any other designation.

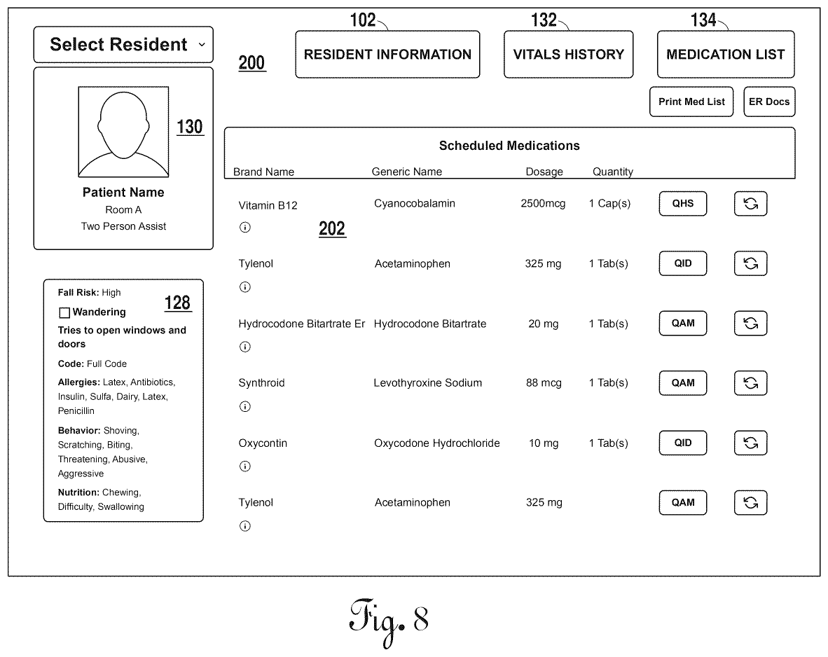

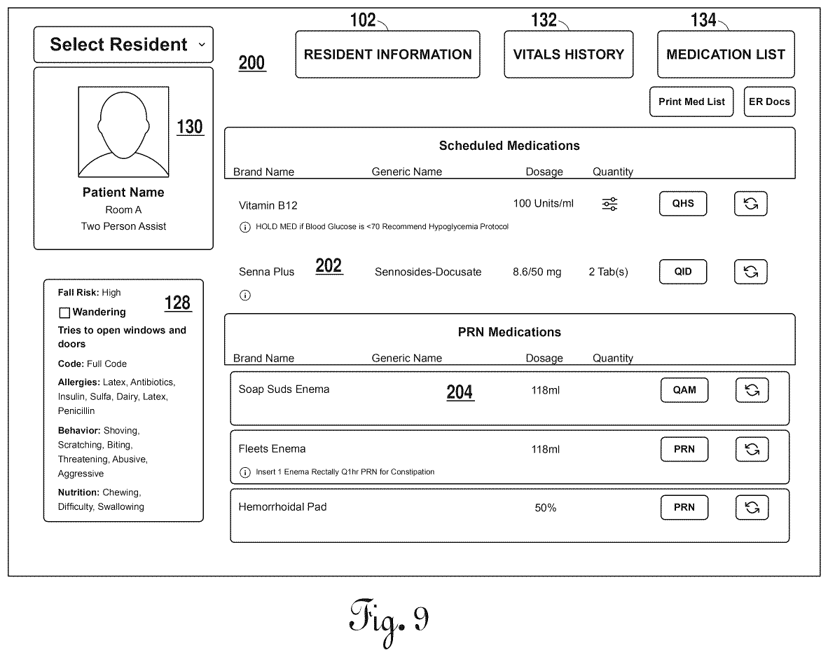

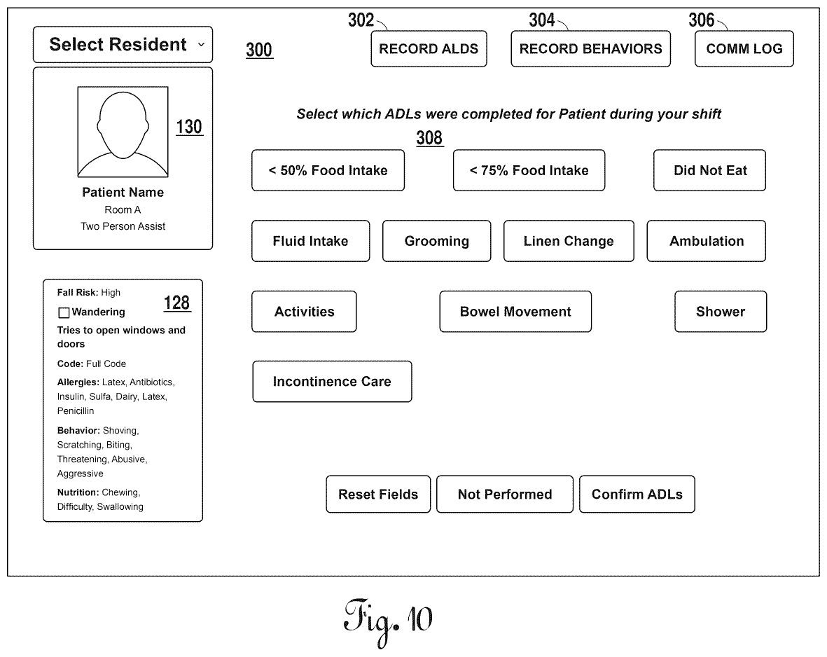

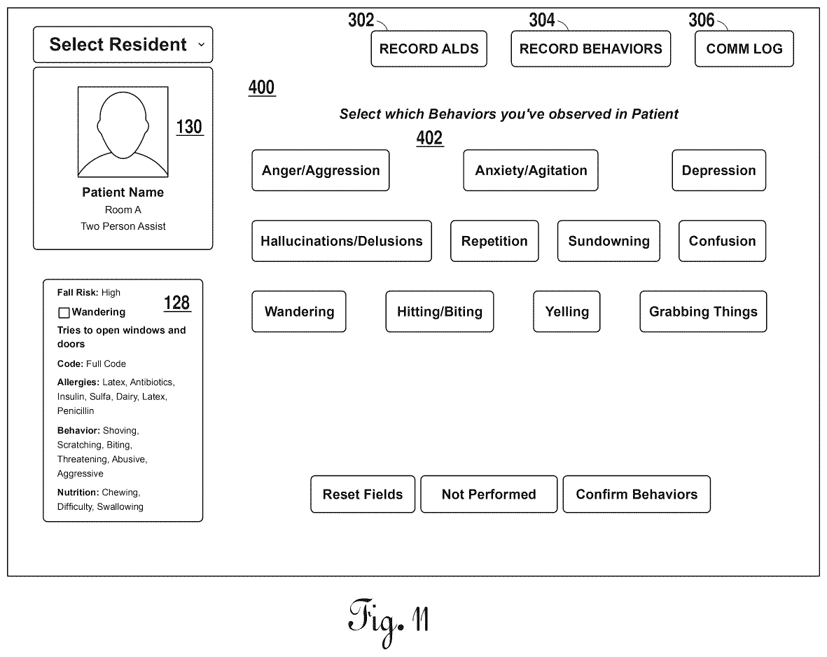

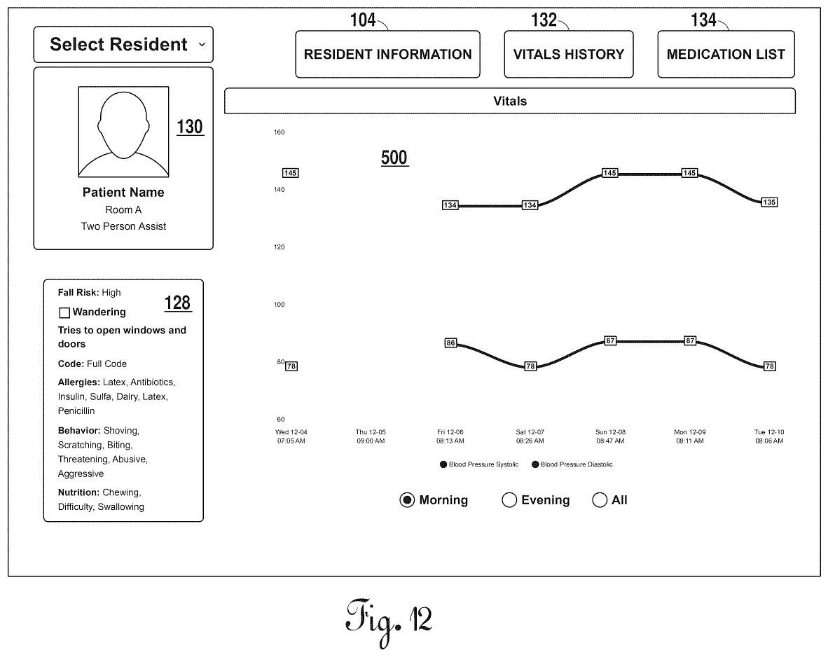

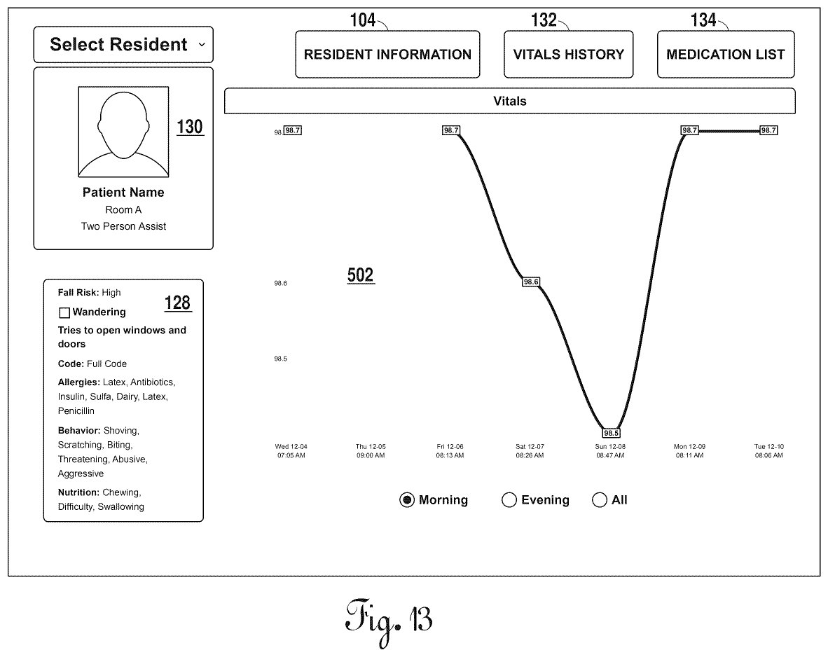

The eMAR module 12 associates a resident page 100 with each resident and each room within the facility. The resident page 100, in one embodiment, is a single resident page with several subpages to record resident information 102, vitals history 132, medication list 134, and if associated with other modules such as ADL module 16, other subpages such as ADL recording 302, recording behaviors 304, communications log 306. Furthermore, if a vitals module 14 is integrated within the system, the resident page 100 and comprises subpages for vitals history 132, medication lists 134. See FIG. 7, FIG. 8, FIG. 9, FIG. 10, FIG. 11, FIG. 12 and FIG. 13.

Still referring to FIGS. 7-13, each resident's page 100 comprises a face sheet 130 which stores relevant information such as name, room number, marital status, date of birth, social security number, gender, religion, and other relevant information. Other relevant information within the resident page 100 that may be stored is primary contact, legal guardian, power of attorney, and other similar relevant information. Furthermore, billing and insurance information, primary care physician information may be stored. Furthermore, within resident page 100 resident preferences such as hospital preference, funeral home preference, pharmacy, and other personal preferences may be stored (not shown).

Activities of Daily Living are basic tasks that individuals typically perform daily. ADLs are used to measure a person's functionality and help determine their need for assistance. The ADL module 16 of the present invention 10 is designed to record, through graphic user interface, via the PCA, six standard ADLs. The six most widely recognized ADL's, which are recorded within ADL module 16, are bathing, dressing, toileting, transferring, eating and incontinence. The ADL module 16 is assigned, on a resident basis, to the PCA caring for that resident, and information on ADLs are entered by the PCA via iPad, tablet, or other device connected to the system of the present invention 10, or if the ADL is in communication with vitals module 14, or a stand-alone ADL module 16, within its own memory.

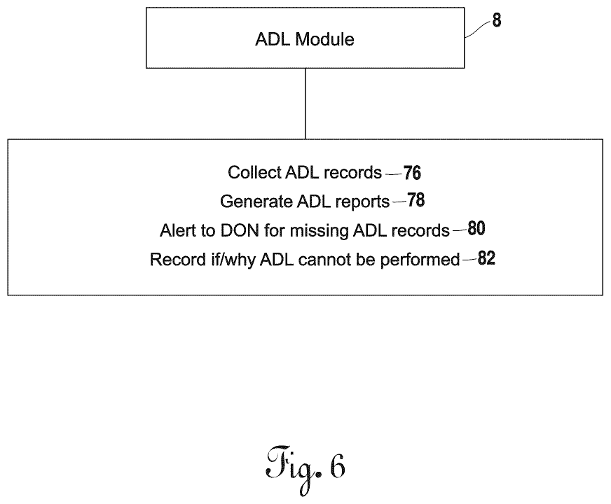

Referring to FIG. 6, ADL module 16 executes commands to collect ADL records 76 when input via graphic user interface by the PCA. ADL module 16 may, upon request from a DON, user, or, if not restricted, PCA to generate ADL report 78. If ADL reports 78 are missing, ADL module 16 executes a command to alert the DON for missing ADL records 80. In this way, an alert system is in place to ensure that the PCA records all daily living activities performed by a resident. If the PCA is unable to input the daily activity of the resident into the ADL module, the PCA may enter an explanation, and a record command within ADL module 16.

Referring to FIGS. 10 and 11, examples of a resident page 100 wherein the subpage record ADLs 302 is displayed. Referring to FIG. 10, the PCA can indicate via GUI several parameters of the six daily activities recorded by ADL module 16. For instance, a resident who has taken in less than 50% of their food can be noted. If the resident was able to groom themselves, move, bathe, and do any of the other daily living activities, the PCA can indicate which ones were performed and which ones were not. Furthermore, referring to FIG. 11, behavioral parameters are set up within ADL module 16 so that the PCA can record within ADL 16 any behaviors observed by the PCA. For instance, if a resident begins hitting or biting the PCA, the PCA can record through their resident screen 100 any such activity.

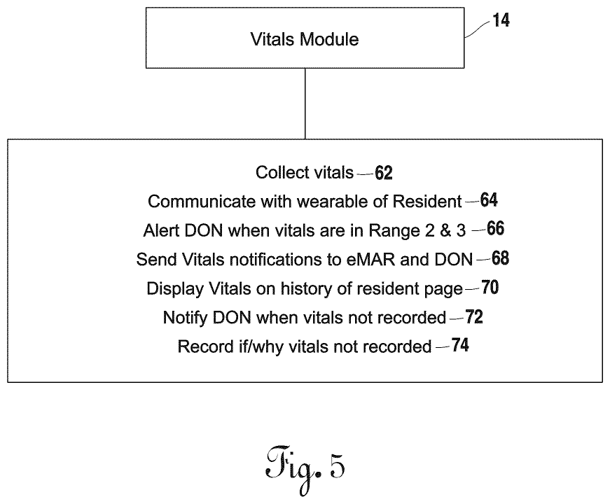

Turning to FIG. 5, vitals module 14 is designed to collect the vitals of each resident The commands within the vitals module 14 allow the vitals module 14 to collect vitals 62. Importantly, in one embodiment, the vitals are taken by the PCA and input into vitals module 14 via graphic user interface through the resident screen 100 in the same manner in which daily activities in behavioral observations are input into the ADL module 16. However, in the preferred embodiment, a wearable 18 is placed on a resident to record the vital signs. There are seven standard vital signs that are checked and monitored by vitals module 14. The vitals module 14 of the present invention 10 is configured to record blood pressure, heart rates, respiration rate, body temperature, blood sugar, blood oxygen and weight. However, if desired by the facility or required in various other installations, fewer or more vital signs may be recorded.

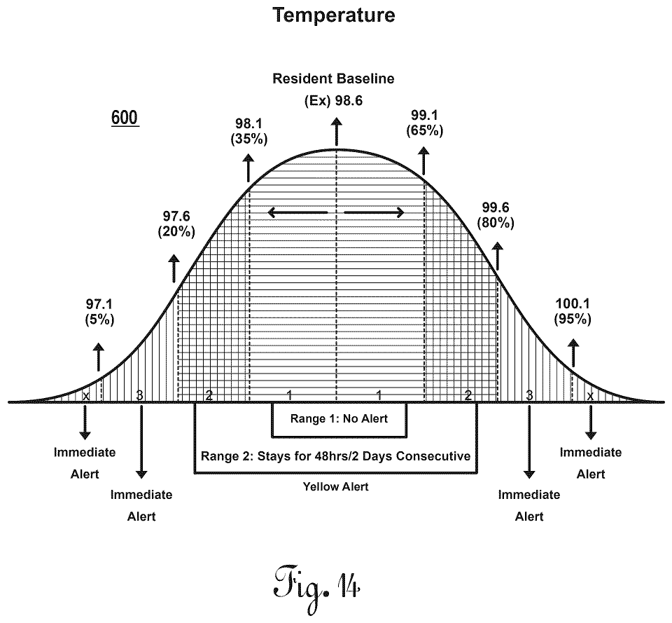

An important aspect of the present vitals module 14 that is not performed by other eMAR systems in the prior art is the vitals module 14 records a seven-day rolling average of each resident's vital signs. For instance, referring to FIG. 14, a graph 600 of a hypothetical resident's temperature is displayed. Graph 600 displays three ranges of body temperature for the resident. Based on a rolling seven-day average, the hypothetical resident has a baseline temperature range of 98.1° to 99.1°. This is a rolling seven-day average indicating that within Range 1, the patient is within normal limits.

However, if a resident has a temperature that begins to fall below or rise above Range 1 indications over the previous seven-day rolling average, vitals module 14 will issue an alert command to alert the PCA and/or DON that the patient is in a moderate risk of being outside of their normal seven-day rolling average. The system 10 displays a visual cue on the resident page 100 to alert the MED Tech and an internal system alert is sent to the DON. Further, if the patient's temperature deviates below 97.6 or above 99.6, which is within Range 2, a Range 3 immediate alert command is executed by vitals module 14, which sends the alert to the DON and MED Tech that the patient is in risk of health consequences due to the range parameter, and text message alerts are sent by the system 10 to the DON, MED Tech and other users responsible for the care of that resident. In this way, as for all of the vitals detected by wearable 18 and recorded by vitals module 14, Range 2 alerts notify the MED Tech and DON that the resident is in potentially increased risk of fall or injury, and Range 3 alerts and text messages alert the MED Tech and DON that medical attention is required, as the resident is at substantially higher risk of fall or injury. Moreover, if a Range 2 or Range 3 cue or alarm is detected, the MED Tech is instructed to manually retake the vital to confirm whether or not the detection is accurate.

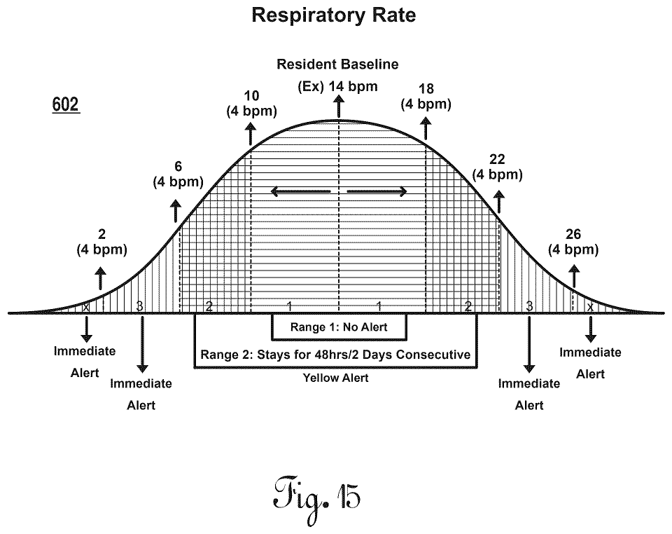

Referring to FIG. 15, another example of a seven-day rolling average for respiratory rate is disclosed. As shown in the graph 602 in FIG. 15, the established seven-day rolling average of 14 breaths per minute of the hypothetical resident is shown on the midline of the bell curve. A deviation of four breaths per minute more or four breaths per minute less indicates a Range 1 deviation wherein no alert is issued by the vitals module 14. However, an increase or decrease of more or less than 4 breaths per minute triggers a Range 2 alert to alert the MET Tech and DON that the resident is at an increased risk of potential fall or injury. A deviation of more or less than eight breaths per minute will trigger a Range 3 alert within the vitals module wherein the DON will be immediately alerted by the command executed by vitals module 14 to indicate that immediate attention is needed for the patient.

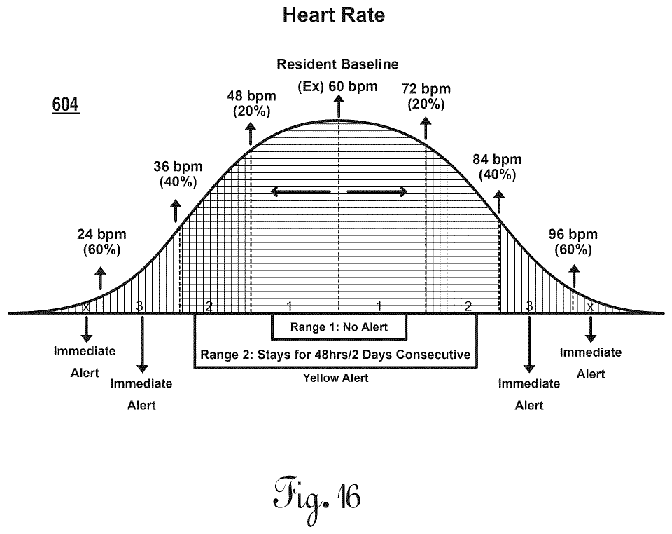

Referring to FIG. 16, a graph showing ranges for a hypothetical resident for heart rate is shown. The seven-day baseline for the hypothetical resident is 60 beats per minute. Deviation of more or less than 12 beats per minute remains within the seven-day rolling average and no alert is triggered by vitals module 14. However, deviation of 13-24 beats per minute above or below the rolling baseline establishes a Range 2 alert command within the vitals module 14, which sends visual cues to resident page 100 to alert the MED Tech, and an alert to the DON. Deviation further than 24 beats per minute above or below the established baseline triggers a Range 3 immediate alert within the vitals module 14, issuing visual cues and alerts with in the system 10 and text message alerts to the DON and MED Tech that immediate medical care is required.

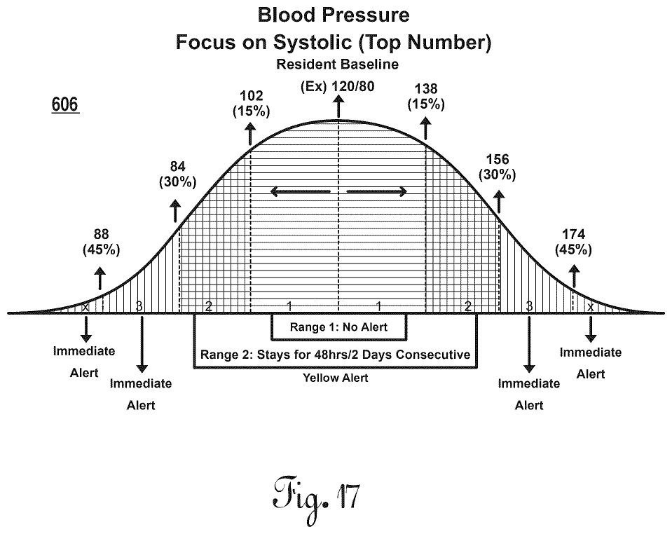

Referring to FIG. 17 graph 606 shows the range notifications and seven-day rolling average for blood pressure for the hypothetical resident. The baseline over the previous seven days is 120/80. Range 2 notifications are alerted at more than 15% deviation from the baseline above or below average, which again causes the vitals module 14 to send visual cues to resident page 100 and notification to the MED Tech and DON. Range 3 immediate alerts are triggered and executed by the vitals module 14 at more than 30% deviation from the baseline, which issues alerts to the DON and MED Tech within the system 10, and text message alerts to the DON and MED Tech that immediate medical care is required.

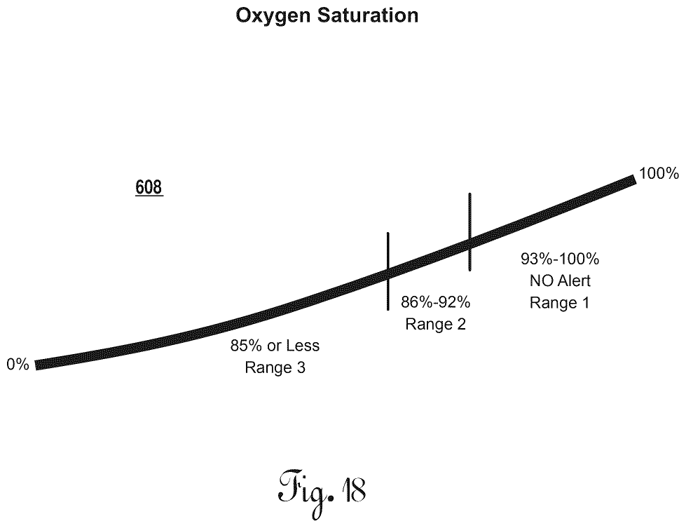

Referring to FIG. 18, a linear graph is displayed for oxygen saturation. In this instance, graph 608 represents a reading wherein any reading between the range of 93-100% oxygen saturation level is considered acceptable, and no alert is triggered within the vitals module 14. Deviation from 86-92% oxygen saturation triggers a Range 2 alert command within the vitals module 14 and will alert the PCA, and/or DON. Deviation below 86% triggers a range 3 warning within the vitals module 14 and will alert the DON immediately.

Referring to FIGS. 12 and 13, examples of vital history 132 recording various of the seven vitals is shown. The seven-day rolling averages 500 in FIGS. 3 and 502 in FIG. 13 are shown.

In the preferred embodiment, each resident is outfitted with a wearable 18. Wearable 18 is preferably a hardware device that is in communication with vitals module 14. In one embodiment, the resident wearable 18 is a wristband that is attached to resident that continuously monitors Blood Pressure, Heart Rate, Oxygen Saturation, Respiratory Rate, Body Temperature) within a given time parameter set forth by the eMAR module 12 in communication with vitals module 14. However, it should be understood that wearable 18 could take other forms, such as an electronic adhesive that is attached to the skin of the resident to perform the functions of collecting the vital statistics of a resident. In this way, vital signs of each resident are automatically taken and collected without need of manual entry through eMAR module 12 or vitals module 14, thereby eliminating human error by the PCA in performing the task, or failure by the PCA to perform the task.

With the vital signs automatically being taken, the vitals module 14 automatically creates seven-day rolling averages of each resident based on the data collected, this in time allows for the Range 2 and range 3 triggers for each vital. The data collected by the vitals module 14 is sent through dependency to eMAR module 12, where it is stored. Moreover, vital dependent medications, such as blood pressure medication, heart rate medication, blood sugar medication, and other sorts of medicine designed to control the vital signs of the resident are cross associated with the data retrieved by wearable 18 and sent to eMAR module 12 through vitals module 14. In this way, if an alert is received by a DON, the DON may associate the vitals dependent medication and order appropriate action for the resident upon receipt of a range 3 warning.

The embodiments described herein are some examples of the current invention it will be appreciated by those skilled in the art that changes could be made to the embodiments in features described above without departing from the broad inventive concept of the present invention. It is understood, therefore, that this invention is not limited to the particular embodiments disclosed, but is intended to cover modifications within the spirit and scope of the present invention. Among other things, any feature described for one embodiment may be used in any other embodiment. Also, unless the context indicates otherwise, it should be understood that when a component is described herein as being connected to another component, such connection may be direct, indirect, and with or without one or more intermediate components. The scope of the invention is defined by the attached claims and other claims that may have been drawn to this invention and is not limited to specific examples described here.

We claim:

1. A vital sign monitoring and alert system comprising:
a vitals monitoring and detection hardware device for detecting vital signs of a person, said device comprising a detection unit for detecting said vital signs and a communication unit for communicating said vital signs,
wherein said device is in constant communication with a vitals detection software system comprising a vitals receiving module for receiving vital signs from said device, a recordation and calculation module for recording and calculating baseline readings of vital signs of a person, a first deviation module, a second deviation module and a third deviation module, a visual cue and alert module and an alarm and text message module, said modules being fixed in a computer readable medium and executed by said vitals detection software system.

2. The vital sign monitoring and alert system as set forth in claim 1 wherein said device is worn on said person's wrist to detect said vital signs through said detection unit.

3. The vital sign monitoring and alert system as set forth in claim 1 wherein said device is attached and adhered to said person's arm to detect said vital signs through said detection unit.

4. The vital sign monitoring and alert system as set forth in claim 2 wherein:
said vitals receiving module is in constant communication with said device to receive vital signs, and is in constant communication with said recordation and calculation module; and
said recordation and calculation module records vital signs received from said receiving module and calculates baseline readings of vital signs for said person, said calculation module calculating said baseline readings based on said vital signs for the previous seven days.

5. The vital sign monitoring and alert system as set forth in claim 4 wherein said first deviation module receives vital signs from said vitals receiving module within a predefined first deviation range.

6. The vital sign monitoring and alert system as set forth in claim 5 wherein said second deviation module receives vital signs from said vitals receiving module within a predefined second deviation range, and communicates an activation instruction to said visual cue and alert module to send visual cues and alerts within said vital sign monitoring and alert system of said second deviation range.

7. The vital sign monitoring and alert system as set forth in claim 6 wherein said third deviation module receives vital signs from said vitals receiving module within a predefined third deviation range, and communicates an activation instruction to said alarm and text message module send alarm signal within said vital sign monitoring and alert system and text messages from said vital sign monitoring and alert system to smart devices to alert of said third deviation range.

8. An electronic medication administration record (eMAR) system comprising:
an eMAR module comprising a plurality of medicine cart assignments, said eMAR module recording a predefined data set of medication administration instructions, a set of alert instructions to send alerts when medications are missed, and a graphic user interface for recording administration of medications;
an ADL module recording a predefined data set for activities for daily living, a set of alert instructions to send when activities for daily living are not performed, and a graphic user interface for recording accomplished or not accomplished activities for daily living, and
a vitals module recording a predefined data set for vital signs, and in constant communication with a monitoring and detection hardware device for detecting vital signs of a person, said device comprising a detection unit for detecting said vital signs and a communication unit for communicating said vital signs,
wherein said device is in constant communication with said vitals module, said vitals module comprising a vitals receiving module for receiving vital signs from said device, a recordation and calculation module for recording and calculating baseline readings of vital signs of a person, a first deviation module, a second deviation module and a third deviation module, a visual cue and alert module and an alarm and text message module, said modules being fixed in a computer readable medium and executed by said vitals detection software system;
wherein said eMAR module, said ADL module and said vitals module are in communication with one another.

9. The eMAR system as set forth in claim 8 wherein said device is worn on said person's wrist to detect said vital signs through said detection unit.

10. The eMAR system as set forth in claim 8 wherein said device is attached and adhered to said person's arm to detect said vital signs through said detection unit.

11. The eMAR system as set forth in claim 8 wherein:
said vitals receiving module is in constant communication with said device to receive vital signs, and is in constant communication with said recordation and calculation module; and
said recordation and calculation module records vital signs received from said receiving module and calculates baseline readings of vital signs for said person, said calculation module calculating said baseline readings based on said vital signs for the previous seven days.

12. The The eMAR system as set forth in claim 11 wherein said first deviation module receives vital signs from said vitals receiving module within a predefined first deviation range.

13. The eMAR system as set forth in claim 12 wherein said second deviation module receives vital signs from said vitals receiving module within a predefined second deviation range, and communicates an activation instruction to said visual cue and alert module to send visual cues and alerts within said vital sign monitoring and alert system of said second deviation range.

14. The eMAR system as set forth in claim 13 Patent wherein said third deviation module receives vital signs from said vitals receiving module within a predefined third deviation range, and communicates an activation instruction to said alarm and text message module send alarm signal within said vital sign monitoring and alert system and text messages from said vital sign monitoring and alert system to smart devices to alert of said third deviation range.

15. An electronic medication administration record (eMAR) system comprising:
an eMAR module comprising a plurality of medicine cart assignments, said eMAR module recording a predefined data set of medication administration instructions, a set of alert instructions to send alerts when medications are missed, and a graphic user interface for recording administration of medications; and
an ADL module recording a predefined data set for activities for daily living, a set of alert instructions to send when activities for daily living are not performed, and a graphic user interface for recording accomplished or not accomplished activities for daily living, and
wherein said eMAR module and said ADL module are in communication with one another.

16. An electronic medication administration record (eMAR) system comprising:
an eMAR module comprising a plurality of medicine cart assignments, said eMAR module recording a predefined data set of medication administration instructions, a set of alert instructions to send alerts when medications are missed, and a graphic user interface for recording administration of medications; and
a vitals module recording a predefined data set for vital signs, and in constant communication with a monitoring and detection hardware device for detecting vital signs of a person, said device comprising a detection unit for detecting said vital signs and a communication unit for communicating said vital signs,
wherein said device is in constant communication with said vitals module, said vitals module comprising a vitals receiving module for receiving vital signs from said device, a recordation and calculation module for recording and calculating baseline readings of vital signs of a person, a first deviation module, a second deviation module and a third deviation module, a visual cue and alert module and an alarm and text message module, said modules being fixed in a computer readable medium and executed by said vitals detection software system;
wherein said eMAR module, said ADL module and said vitals module are in communication with one another.
